(19) United States
(12) Patent Application Publication
(10) Pub. No.: US 2013/0149703 A1
(43) Pub. Date: Jun. 13, 2013

(54) "MARKERS FOR PROSTATE CANCER PROGRESSION"

(71) Applicants: Eric Levesque, Quebec (CA); Chantal Guillemette, Quebec (CA); Yves Fradet, Quebec (CA); Louis Lacombe, L'Ancienne-Lorette (CA)

(72) Inventors: Eric Levesque, Quebec (CA); Chantal Guillemette, Quebec (CA); Yves Fradet, Quebec (CA); Louis Lacombe, L'Ancienne-Lorette (CA)

(73) Assignee: UNIVERSITE LAVAL, Quebec, QC (CA)

(21) Appl. No.: 13/701,163

(22) PCT Filed: May 31, 2011
(86) PCT No.: PCT/CA2011/050326

§ 371 Date: Feb. 11, 2013

Related U.S. Application Data
(60) Provisional application No. 61/350,148, filed on Jun. 1, 2010.

(51) Int. Cl. C12Q 1/68

(52) U.S. Cl. CPC C12Q 1/6886
USPC 435/6.11

(57) ABSTRACT

Purpose. The relationship between inherited genetic variations in 5α-reductase type 1 (SRD5A1) and type 2 (SRD5A2) genes and the risk of biochemical recurrence after radical prostatectomy (RP) in prostate cancer (PCa) remains a fairly unexplored area of research. Patients and Methods. We studied 526 men with organ-confined and locally advanced PCa with a median follow-up time of 7.4 years. We investigated the effects of allelic variants of SRD5A1 and SRD5A2 genes and haplotype-tagging single nucleotide polymorphisms (htSNPs; n=19) on recurrence-free survival after RP using Kaplan-Meier plots, the log-rank test, and Cox proportional hazard models. Results. Upon adjusting for known prognostic clinical and pathological factors, eight htSNPs were shown to be independent predictors of recurrence. The SRD5A1 rs166050 polymorphism was associated with an increased recurrence risk of HR=1.83 (95% CI, 1.04-3.21; P=0.035), while the rs518673 in SRD5A1 was associated with a decreased risk (HR=0.59, 95% CI, 0.41-0.85; P=0.004). The SRD5A2 gene was strongly associated with the risk of relapse with six polymorphisms being positively associated with recurrence including the known SRD5A2 V89L (rs523349) (HR=2.14, 95% CI, 1.23-3.70; P=0.007) and a protective htSNP rs12470143 with a HR of 0.66, (95% CI, 0.46-0.95; P=0.023). By combining SRD5A1 (rs518673T) and SRD5A2 (rs12470143 A), the protective effect was shown to be additive with the maximum protection conferred by 3 or 4 alleles (HR=0.33, 95% CL 0.17-0.63; P=0.001). Conclusion. Germline polymorphisms in 5α-reductase genes are independent prognostic genetic biomarkers that predict PCa biochemical recurrence after radical prostatectomy and may represent useful molecular tools for a genotype-tailored clinical approach.

### FIELD OF THE INVENTION

The present invention relates to markers for the prognosis of recurrence of prostate cancer.

### BACKGROUND OF THE INVENTION

Prostate Cancer (PCa) is a major public health concern since it is the sixth most common cancer in the world and the second leading cause of cancer death in North American men.¹ Of the several known risk factors, the most important are age, ethnicity, dietary and genetic factors.²⁻⁴ Patients with localized (clinical stage T1-T2) and locally advanced (T3) PCa are frequently treated with radical prostatectomy (RP), a potentially curative procedure. It is estimated that over 30% of men undergoing RP will have disease relapse, also referred to as biochemical recurrence (BCR) as the first clinical indication of rising serum level of PSA.⁵

Currently, the Tumor/Nodes/Metastasis (TNM) staging system, the Gleason score, and pre-treatment serum prostate-specific antigen (PSA) are the most important factors influencing both the likelihood of more extensive disease and the probability of subsequent relapse following RP.^{6, 7} Indeed, these tools include both nomograms and risk tables incorporating clinical variables that can predict, although still imperfectly, the likelihood of tumor recurrence and provide crucial prognosis information to guide clinicians in their therapeutic decisions. The risk of disease progression greatly differs between individuals and the heterogeneity in clinical behaviour further emphasizes the need to find novel markers of progression. Even if cases of PCa are considered localized at the time of diagnosis, the rate of BCR after RP is still significant and occur most often in the 5 years after surgery.⁸ The persistence of tumor cells in a state of either complete or near dormancy prior to metastatic progression is likely accountable for disease recurrence while these residual cells are most probably responsive to hormones.

Androgen hormones, such as testosterone (T) and 5α-dihydrotestosterone (DHT), have been clearly implicated in development of PCa. Circulating T, secreted by the testis, and adrenal steroid precursors (dihydroepiandrosterone; DHEA and DHEA-sulfate; DHEA-S) are among factors influencing androgen levels in the prostate and other tissues.⁹⁻¹¹ Prostate cells, as well as a number of peripheral tissues, contain a variety of steroidogenic enzymes required for the local formation of active androgens from adrenal precursors.¹²⁻¹⁴⁻¹⁵ Namely, the conversion of T by 5α-reductases (SRD5A1 and SRD5A2) leads to DHT, the more potent androgen receptor (AR) agonist in target cells. SRD5A2 is the major 5α-reductase enzyme expressed in the prostate compared to SRD5A 1.¹⁶ However, while the expression of SRD5A2 decreases in prostate cancer cells, SRD5A1 is increased in tumoral tissues.¹⁶⁻²⁰ This imbalance in the expression of SRD5A genes in PCa tumors illustrate the complex relation between 5α-reductases, DHT synthesis and PCa progression.

Androgen deprivation therapy (ADT) is the standard of care for metastatic PCa and is also used to treat asymptomatic patients with PSA recurrence after failed primary therapy (RP), further reinforcing the initial androgen dependency of these cells.^{21, 22} Finasteride, a 5α-reductase type 2 inhibitor currently used in the clinic, has been recently shown to be an effective chemopreventive medication reducing by almost 25% the risk of PCa incidence.²³ Additionally, data from clinical studies were recently used to model a risk-adapted PSA-based chemoprevention strategy.²⁴ Despite this well recognized hormonal dependence of prostate cancer cells in the early cancer stage, very few studies have investigated the associations between polymorphisms in the androgen biosynthesis pathway and clinical outcome after surgical procedure.²⁵⁻³² To date, common polymorphisms such as those in sex-steroids biosynthesis pathways have been extensively studied in relation to risk of PCa.^{19, 33-43} However, almost all of these studies did not address the association between polymorphisms in genes regulating hormonal exposure with PCa recurrence and survival, and were not designed to do so. Long-term longitudinal studies are thus still required to systematically evaluate the impact of a patient's genetic profile on risk of recurrence.

We hypothesize that variations in SRD5A genes may alter systemic androgen availability and affect the tumoral microenvironment exposure to hormones, which could modify the risk of PCa recurrence after RP. This is based on the fact that 5α-reductases have a well characterized physiological role in DHT biosynthesis, are associated with PCa risk and more recently, with cancer progression^{26, 28} and represent molecular targets in PCa prevention trials^{23, 24}. In this study, we performed a detailed genetic analysis of the SRD5A1 and SRD5A2 genes in relation to PCa progression. We also aimed at validating the association of the known SRD5A2 V89L polymorphism associated with BCR by other groups.^{26, 28} One particular feature of our study is that the association of inherited variations with the risk of BCR was determined after RP as the sole initial curative intent in a cohort of 526 men with clinically localized and locally advanced PCa.

### SUMMARY OF THE INVENTION

There is therefore provided an in-vitro method for providing a diagnosis, prognosis or predicting the likelihood of a human subject to develop prostate cancer or a recurrence thereof, said method comprising the steps of: a) obtaining a nucleic acid from a nucleic acid-containing sample (particularly a non-tumor or a tumor sample) from said human subject; and b) determining the individual's genetic variations (or haplotypes) in SRD5A1 or SRD5A2 gene in comparison to normal sequence of said genes; whereby the presence of at least one genetic variation in SRD5A1 or SRD5A2 in said subjects nucleic acid is an indication that said subject has an increased or a decreased likelihood that the prostate cancer will develop or recur.

The invention further provides the method as defined herein wherein step b) further comprises the step of: b′) identifying at least one single nucleotide polymorphism (SNP) in said nucleic acids, said SNP being selected from the group consisting of: rs518673; rs166050; rs12470143; rs2208532; rs2300702; rs4952197 and rs676033 or any of their associated variants as listed in Table 3; whereby the presence of at least one of said markers in said subjects sample is an indication that said subject has an increased or a decreased likelihood that the prostate cancer will develop or recur.

The present invention also provides a method for adapting a course of treatment of prostate cancer in a human subject after the subject has undergone radical prostatectomy, comprising the steps of: a) providing a prognosis or predicting the likelihood of a human subject to develop prostate cancer recurrence in accordance with the method as defined herein; and b) adapting a course of treatment according to whether said subject has an increased or decreased likelihood that the cancer will recur.

The invention further provides a kit for predicting the likelihood of a human subject to develop prostate cancer and/or recurrence by detecting a SNP in a reference sequence selected from the group consisting of: rs518673; rs166050; rs12470143; rs2208532; rs2300702; rs4952197 and rs676033 or their associated SNPs; said kit comprising reagents for determining the individual's genetic variations (or haplotypes) in SRD5A/or SRD5A2 gene.

### DETAILED DESCRIPTION OF THE INVENTION

### Description of the Figures

FIG. 1 shows the risk of recurrence associated with known clinical and pathological prognostic variables (A) and SRD5A genes (B). Boxes represent hazard ratios (HR) and their 95% CI. PSA categories are in ng/ml. Reference categories (HR: 1.00) are: PSA at diagnosis ≦10 ng/ml, pG≦6, and pT≦T2b. Genetic linkage between htSNPs tested for each SRD5A gene is represented in the triangles on the left in panel B; and

FIG. 2 illustrates Kaplan-Meier estimates of recurrence-free survival for A) SRD5A1, B) SRD5A2 and C) both genes. Only positive htSNPs in multivariate analysis are represented. Values for log-rank P values (LR) are shown in each frame. Numbers (0 to 4) in panel C indicate the number of protective alleles for both genes. SRD5A1 protective allele is rs518673T and SRD5A2 protective allele is rs12470143A.

### DEFINITIONS AND ABBREVIATIONS

ADT: androgen-deprivation therapy; AR: androgen receptor; BCR: biochemical recurrence; DHT: 5α-dihydrotestoterone; HR: hazard ratio; htSNP: haplotype-tagging SNP; PCa: prostate cancer; PSA: prostate-specific antigen; SRD5A 1: 5α-reductase type 1; SRD5A2: 5α-reductase type 2; RP: radical prostatectomy; SNP: Single nucleotide polymorphism; T: Testosterone.

### DETAILED DESCRIPTION OF PARTICULAR EMBODIMENTS

In accordance with the method of the invention, it will be well recognized by persons skilled in the art that genetic variations are assessed in comparison to the gene sequence identified for the normal gene. Such gene sequence for each of these enzymes in their normal state can be found at:
for SRD5A1: Ensembl accession number ENSG00000145545 and can be consulted from the NCBI Internet site at the reference number: gene ID 6715.
for SRD5A2: Ensembl accession number ENSG00000049319 and can be consulted from the NCBI internet site at the reference number: gene ID 6716.

In accordance with particular aspects of the present invention, step b) of the method may further comprise: b″) contacting the subjects nucleic acid with a reagent that specifically binds to at least one of said single nucleotide polymorphism (SNP); and c) detecting the binding of reagent to at least one of said SNP, whereby the binding of said reagent to at least one SNP is an indication that said subject has an increased or a decreased likelihood that the prostate cancer will recur.

The invention also provides the method as defined herein, wherein the SNP is found in reference sequences (rs) selected from the group consisting of: rs166050; rs2208532; rs2300702; rs4952197; and rs676033, or any of their associated SNPs whereby the presence of said SNP is an indication of an increased likelihood that the prostate cancer will recur. Particularly, the SNP is found in reference sequences (rs) selected from the group consisting of: rs2208532 or rs676033, whereby the presence of said SNP is an indication of an increased likelihood that the prostate cancer will recur. More particularly, the SNP is found in rs2208532 or any of its associated SNPs and the presence of said SNP is an indication of an increased likelihood that the prostate cancer will recur.

The invention also provides the method as defined herein, wherein the SNP is found in reference sequences (rs) selected from the group consisting of: rs518673 or rs12470143, whereby the presence of said SNP is an indication of a decreased likelihood that the prostate cancer will recur. Particularly, the presence of SNP found in rs518673 or any of its associated SNPs is an indication of a decreased likelihood that the prostate cancer will recur. More particularly, the presence of both rs518673T and rs12470143A or any of their associated SNPs is still a further indication of a decreased likelihood that the prostate cancer will recur.

The invention also provides the method as defined herein wherein in step a), the nucleic acid is DNA or RNA. In particular embodiment of the invention, the DNA is extracted from a non-tumor or a tumor sample from said human subject to be utilized directly for identification of the individual's genetic variations. Particularly, examples of nucleic acid detection methods are: direct sequencing or pyrosequencing, massively parallel sequencing, high-throughput sequencing (a.k.a next generation sequencing), high performance liquid chromatography (HPLC) fragment analysis, capillarity electrophoresis and quantitative PCR (as, for example, detection by Taqman® probe, Scorpions™ ARMS Primer or SYBR Green),In one aspect, the amplification of the DNA is carried out by means of PCR. Several methods for detecting and analyzing the PCR amplification products have been previously disclosed. The general principles and conditions for amplification and detection of genetic variations, such as using PCR, are well known for the skilled person in the art.

Alternatively, other methods of nucleic acid detection such as hybridization carried out using appropriately labeled probe, detection using microarrays e.g. chips containing many oligonucleotides for hybridization (as, for example, those produced by Affymetrix Corp.) or probe-less technologies and cleavage-based methods may be used. Preferably, amplification of the DNA can be carried out using primers that are specific to the marker, and the amplified primer extension products can be detected with the use of nucleic acid probe. More particularly, the DNA is amplified by PCR prior to incubation with the probe and the amplified primer extension products can be detected using procedure and equipment for detection of the label.

The invention also provides the method as defined herein, wherein the subject's tumor sample is from a biopsy. The invention also provides the method as defined herein, wherein the subject's non-tumor sample is selected from the group consisting of: tissue or biological fluid. Particularly, the tissue is a lymph node, hair or a buccal smear. Still, particularly, the biological fluid is sputum, saliva, blood, serum urine, semen or plasma.

The invention also provides the method as defined herein, wherein, when the likelihood of recurrence is increased, the subject is prescribed 5α-reductase inhibitors therapy.

The invention also provides the kit as defined herein, comprising PCR primer-probe set, wherein the primers are selected from the group consisting of: SEQ ID Nos. 1 to 38; and the probe is selected from the group consisting of: SEQ ID Nos. 39 to 57.

### EXAMPLES

### Patients and Methods

### Clinical Data and Outcome Collections

The study cohort, mostly composed of Caucasians, included 526 men who underwent RP at l'Hôtel-Dieu de Québec Hospital (QC, Canada) between February 1999 and December 2002. Each participant provided written consent before surgery for the analysis of their genome and the research protocol was approved by the research ethical committee at the Centre Hospitalier Universitaire de Québec (CHUQ, QC, Canada). All patients were followed postoperatively with serial PSA measurements and detailed clinical information was available.

### DNA Isolation and Genetic Analysis

Polymorphisms studied were chosen according to one or more of the following criteria: i) to be likely functional (with supportive data in the literature), ii) to have previously been associated with PCa risk, aggressiveness, age at onset, BCR or ADT efficiency, and iii) to explain most of the haplotype diversity in the CEU (Utah residents with Northern and Western European Ancestry) Hapmap population. For both SRD5A genes, a region covering all the exons, introns and 5 kb of the 5′ and 3′ sections of the genes was screened using a haplotype tagging SNPs (htSNPs) strategy to maximize coverage, using HapMap Phase 2 (www.hapmap.og/cgi-perl/gbrowse/hapmap3r2_B36)⁴⁴ and data from the CEU unrelated subjects based on a r2≧0.80 and a minimum minor allele frequency ≧0.05.

Peripheral blood was collected on the morning of a preoperative clinic visit and kept frozen at −80° C. until analysis. Genomic DNA was purified using the QIAamp DNA Blood Mini Kit (Qiagen Inc., Mississauga, ON, Canada) and stored at −20° C. PCR amplifications were performed using Sequenom iPLEX matrix-assisted laser desorption/ionization-time-of-flight mass spectrometry by the sequencing service of McGill University and Génome Québec INNOVATION center (QC, Canada). For oligos sequence, see Table 1. Negative controls were present for every run of analyses and quality controls (random replicates of known genotypes) were successfully performed in 5% of the study cohort.

TABLE 1
Oligonucleotides used for genotyping analysis.

| | | | SEQ | | SEQ |
|---|---|---|---|---|---|
| | | | ID | | ID |
| Gene | SNP | Forward Primer Sequence | No. | Reverse Primer Sequence | No. |
| SRD5A1 | rs518673 | ACGTTGGATGCCGTTTCCCTCATGTTGTTC | 1 | ACGTTGGATGGAGAGAGAATGAGTGCTGAG | 20 |
| | rs166050 | ACGTTGGATGTGTGTATTCTCTGGACACGC | 2 | ACGTTGGATGACCAAAGTAGGTTATCGGGC | 21 |
| | rs8192120 | ACGTTGGATGCCCTCCATCTTTGTAGTTAC | 3 | ACGTTGGATGTTGAGCCCTAAGAGCAACAC | 22 |
| | rs501999 | ACGTTGGATGGGTGCTATCCTAGTCCCATC | 4 | ACGTTGGATGTTCCATCACCATGCCCATTC | 23 |
| | rs500182 | ACGTTGGATGCCCTTGATTTCCAACACCAC | 5 | ACGTTGGATGCATTGCTGTGAATGCACATC | 24 |
| | rs4702378 | ACGTTGGATGACCAAAGTAGGTTATCGGGC | 6 | ACGTTGGATGTGTGTATTCTCTGGACACGC | 25 |
| | rs3822430 | ACGTTGGATGAGCCGTTACAGGTACAGAAC | 7 | ACGTTGGATGTTTCTTGTTTCCTAAGGTGC | 26 |
| SRD5A2 | rs2208532 | ACGTTGGATGTTCTTGAGCTTTGTTCTGGG | 8 | ACGTTGGATGACACTCTTCTGGAACCACTG | 27 |
| | rs523349 | ACGTTGGATGGGGAAAAACGCTACCTGTGG | 9 | ACGTTGGATGTGCAGGAGCTGCCTTCCTTC | 28 |
| | rs676033 | ACGTTGGATGAACAGAAGACACAGCACACC | 10 | ACGTTGGATGTACAGTAAAGGCTCTTGCCC | 29 |
| | rs9332975 | ACGTTGGATGTGGCTACTAAGAGGATAGAG | 11 | ACGTTGGATGAAGCAGCATGTAACCTCATC | 30 |
| | rs2281546 | ACGTTGGATGGTCACATGAGCTAGTTCCAC | 12 | ACGTTGGATGTCTTGCCTGCACCTTTTATC | 31 |
| | rs12470143 | ACGTTGGATGCTCTCATTAAACCTCTCCCG | 13 | ACGTTGGATGTGGAGAATAGATTGCGGTGG | 32 |
| | rs2300702 | ACGTTGGATGTCTACCTCAGCATATCTCCC | 14 | ACGTTGGATGGTTGCAGAAACCTCAGATTG | 33 |
| | rs9282858 | ACGTTGGATGAAGCACACGGAGAGCCTGAA | 15 | ACGTTGGATGAAGGCAGCTCCTGCAGGAAC | 34 |
| | rs4952197 | ACGTTGGATGTGGCACAAGCACTTCCTTAG | 16 | ACGTTGGATGCACAAGGACTGCAATTCCTG | 35 |
| | rs7562326 | ACGTTGGATGCTGAAAAGACAGCAGAAGGG | 17 | ACGTTGGATGTCCTCAGGCAAGGAAATGTG | 36 |
| | rs4952222 | ACGTTGGATGAAGATATGCCCGCTGCTTTG | 18 | ACGTTGGATGGGCCATAGTTTACCAATCCC | 37 |
| | rs3754838 | ACGTTGGATGACTTAGCTGAGGCTCTCATC | 19 | ACGTTGGATGGAGGCCTTGTGTGTTTCTTC | 38 |

| | | | SEQ |
|---|---|---|---|
| | | | ID |
| Gene | SNP | Probe Sequence | No. |
| SRD5A1 | rs518673 | GGAAGGACGAAGCCCCTAATAAACCAT | 39 |
| | rs166050 | GAAGCAAGGAAAGCTGTGCATCAA | 40 |
| | rs8192120 | TGCTCTCCTAAGCATATCCTAC | 41 |
| | rs501999 | GGGGCAGCTTAGTAACTCATCC | 42 |
| | rs500182 | AATGCACATCCAGACA | 43 |
| | rs4702378 | GCTTTCCTTGCTTCAC | 44 |
| | rs3822430 | TCTAAGGTGCTTAATTTACCC | 45 |
| SRD5A2 | rs2208532 | TCAACACTGAACAAATCAGATCAAA | 46 |
| | rs523349 | CTTCTGGGCCTCTTCTGC | 47 |
| | rs676033 | GCTTGACCCCTCACTCC | 48 |
| | rs9332975 | GGTCTCTTTGTTTTACACTACTCATTATTT | 49 |
| | rs2281546 | CAGCAAACGATTTCCAAGGAAGTCCCCC | 50 |
| | rs12470143 | GGCGGTGGAGTAAATGT | 51 |
| | rs2300702 | TAGGGTGCTTTTGTTCTTTTTTTGTTTG | 52 |
| | rs9282858 | AAAGGGAACCAGGCGGCGCGGG | 53 |
| | rs4952197 | GGGCGTGCTGTGCTGGGCTTAGA | 54 |
| | rs7562326 | GAGCAAGGAAATGTGAATTACC | 55 |
| | rs4952222 | CTAAAGAAAACTATGAATGGCGTG | 56 |
| | rs3754838 | TAGTACTTTTGTTATTAACATTATATAGAC | 57 |

### Statistical Analysis

Based on our sample size, we were able to detect a hazard ratio of 1.50, for a minor allele with frequency of 5%, with over 80% statistical power. Allelic frequencies and Hardy-Weinberg equilibrium were computed with PLINK (version 1.07), a free open-source whole genome association analysis toolset.⁴⁵ To analyse their association with BCR, each SNP was considered using 3 models since the function of most htSNP remain unknown. The first model tested, named the genomic model, considered the SNP as a categorical variable with a common allele homozygote (reference; based on the frequent allele reported in the Hapmap project), heterozygote and a minor allele homozygote. The second model, named the dominant model, considered the SNP with only 2 categories: one with a common allele homozygote (reference) and one with at least one minor allele. Finally, the third one referred to as the recessive model, also considered the SNP with only 2 categories: one with at least one common allele (reference) and one minor allele homozygote. Cox regression was performed on each SNP considering the 3 above mentioned models with adjustment for confounding variables namely PSA level at diagnosis, age at diagnosis, smoking status, pathological Gleason grade, pathological stage and neoadjuvant ADT. All co-variables were treated as categorical, and for PSA level, Gleason scores and stage, they were used as described by the well-recognized D'Amico risk classification.⁷ Smoking status and neoadjuvant ADT were classified as “positive” or “negative” while age was classified as <65 and ≧65 years old. The censoring variable was BCR, which was defined as 1) two consecutive PSA values ≧0.3 μg/L, 2) one PSA value ≧0.3 μg/L followed by ADT, 3) a last-recorded PSA value ≧0.3 μg/L, and 4) the initiation of ADT or radiation therapy by the patient's physician. Kaplan-Meier analyses were also processed for every SNP (log rank), while only results for SNPs which were significantly associated with BCR in Cox regression multivariate analysis are shown. For Kaplan-Meier and Cox regression, statistical analyses were performed using PASW statistics 17 (SPSS Inc., Chicago, Ill.) and R version 2.10.0 (http://www.r-project.org/).

Haplotypes were inferred using Phase v2.1.1 program,⁴⁴ and their relative frequency, as well as pairwise linkage disequilibrium between SNPs, were determined with HAPLOVIEW 4.1.⁴⁶ Univariate and multivariate analysis were performed with or without minor haplotypes (frequency<5%), without significant impact on the P values—therefore, only results without minor haplotypes are shown.

### Results

Clinical and pathological characteristics of the study cohort are shown in Table 2. All 526 patients had initially RP as curative intent enabling a precise pathological evaluation. The actual median follow-up time of the cohort is 7.4 years (range: 0.5 to 10.2 years). The cohort had mainly organ-confined and locally advanced tumors, as PCa cases were composed mainly of pT2 (60%) and pT3 (37%) pathological tumor stages (Table 2). Overall, 130 cancer cases experienced BCR (25%), which was our primary outcome variable, with a median time to relapse of 2.1 years.

TABLE 2
Clinical and Pathological Characteristics of the Study Cohort.

| | | |
|---|---|---|
| Characteristic | Number | % |

| | |
|---|---|
| Age at diagnosis, years (n = 526) | |
| Mean | 63.3 |
| Standard deviation | 6.8 |
| Range | 43.5-80.7 |

| | | |
|---|---|---|
| Smoking status (n = 523) | | |
| No | 438 | 84 |
| Yes | 85 | 16 |
| PSA at diagnosis (ng/mL) (n = 521) | | |
| ≦10 | 362 | 69 |
| >10-20 | 103 | 20 |
| >20 | 56 | 11 |
| Pathological Gleason score (n = 509) | | |
| pG ≦ 6 | 158 | 31 |
| pG = 7 | 244 | 48 |
| pG ≧ 8 | 107 | 21 |
| Pathological T stage (n = 522) | | |
| pT ≦ T2 | 314 | 60 |
| pT = T3 | 195 | 37 |
| pT = T4 | 13 | 3 |
| Neoadjuvant hormonotherapy (n = 526) | | |
| Yes | 31 | 6 |
| No | 495 | 94 |
| Biochemical recurrence (n = 526) | | |
| Yes | 130 | 25 |
| No | 396 | 75 |

PSA, prostate-specific antigen;
n, number of patients for which the information was available.

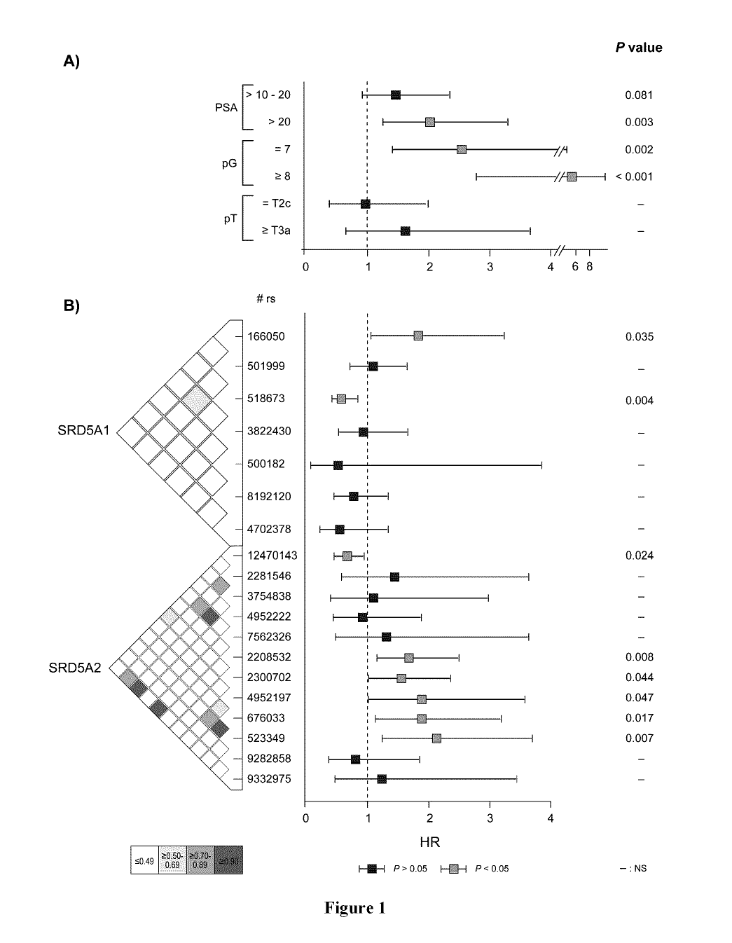

Risk of recurrence associated with known clinical and pathological prognostic variables are shown in FIG. 1A. As expected, the risk of recurrence increase with higher PSA values at diagnosis with a risk of relapse of EIR=1.5 (p=0.081) and EIR=2.1 (p=0.003) with PSA values of 10-20 ng/mL and ≧20 ng/mL, respectively. Gleason scores of 7 and ≧8 are also positively associated with relapse with HR of 2.6 (p=0.002) and 5.4 (p<0.001), respectively. Pathological stage was not associated with biochemical recurrence risk (FIG. 1A).

A total of 19 htSNPs of which 2 functional coding SNPs, distributed across the two SRD5A genes, were studied herein. The htSNPs strategy allowed us to study 89 genetic variations in both genes (Table 3). htSNPs were selected with a strategy to maximize gene coverage and to reflect adequately the Caucasian haplotype genetic diversity.

TABLE 3
List of htSNPs included in this study and their associated SNPs

| Gene | htSNP | Associated SNPs | htSNP | Associated SNPs | htSNP | Associated SNPs | htSNP | Associated SNPs |
|---|---|---|---|---|---|---|---|---|
| SRD5A1 | rs518673 | rs518673 | rs500182 | rs248807 | rs500182 | rs2677947 | rs3822430 | rs8192130 |
| | rs166050 | rs166050 | | rs482121 | | rs248803 | | rs4702375 |
| | | rs471604 | | rs2677933 | | rs166049 | | rs11738248 |
| | | rs824811 | | rs494958 | | rs500182 | | rs7706809 |
| | rs8192120 | rs8192120 | | rs248800 | | rs521293 | | rs7720479 |
| | rs501999 | rs248797 | | rs562461 | | rs568509 | | rs8192166 |
| | | rs248805 | | rs248799 | | rs484973 | | rs8192131 |
| | | rs477930 | | rs500058 | rs4702378 | rs4702379 | | rs7707559 |
| | | rs501999 | | rs1691052 | | rs4702378 | | rs4702374 |
| | | rs566202 | | rs1651074 | | rs1560149 | | rs6884552 |
| | | rs472402 | | rs535981 | rs3822430 | rs3822430 | | rs1896670 |
| | | rs531241 | | rs193744 | | rs11134173 | | rs8192139 |
| | | rs181807 | | rs168713 | | rs3733773 | | |
| SRD5A2 | rs2208532 | rs2208532 | rs676033 | rs522638 | rs2281546 | rs2268794 | rs9332975 | rs6543634 |
| | | rs2300701 | | rs632148 | | rs11889731 | | rs9332975 |
| | | rs765138 | | rs806645 | | rs17011453 | | rs3731586 |
| | | rs559555 | | rs502139 | | rs1042578 | | rs1884722 |
| | | rs2300697 | | rs2754530 | | rs2281546 | | rs7571644 |
| | rs523349 | rs523349 | | rs655548 | | rs11892064 | | rs7589579 |
| | | rs12467911 | | rs623419 | | rs11690596 | rs4952197 | rs4952197 |
| | | rs2300699 | | rs499362 | | rs7567093 | | rs4952218 |
| | | rs558803 | | rs546935 | | rs1090817 | rs3754838 | rs3754838 |
| | | rs612224 | | rs481344 | rs12470143 | rs2300700 | | rs13027103 |
| | | rs477517 | rs2300702 | rs2268796 | | rs481126 | rs9282858 | rs9282858 |
| | | rs682895 | | rs2300702 | | rs585890 | rs7562326 | rs7562326 |
| | rs676033 | rs676033 | | rs2268797 | | rs564310 | rs4952222 | rs4952222 |
| | | rs599300 | | rs4952220 | | rs665237 | | |
| | | rs614173 | rs2300702 | rs2300703 | | rs12470143 | | |

Associated SNPs are polymorphisms in strong linkage with the htSNP with a r² ≧ 0.80. The list of associated SNPs is derived from the analysis of a region covering approximately 250 kb for each SRD5A genes.

After analyses with a Cox regression multivariate model, adjusted for all clinical and pathological factors known to affect BCR, 8 htSNPs were positively associated (P<0.05) with the risk of relapse. Their relative frequencies in cancer patients with and without relapse, and the corresponding hazard ratios (95% CI) are displayed in FIG. 1 and Table 4. Genetic linkage between the 19 htSNPs tested in both SRD5A genes is also represented.

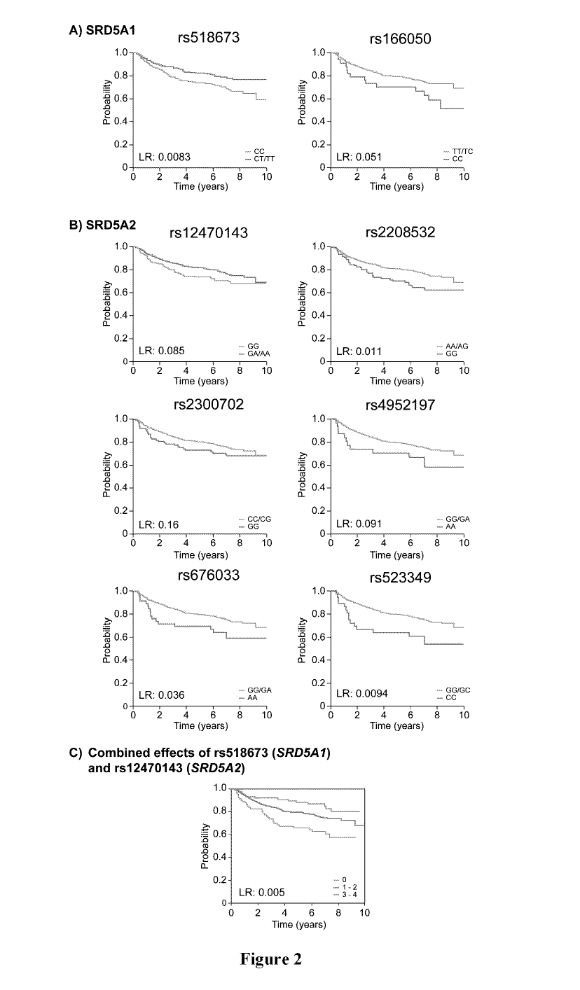

Among 11 htSNPs in SRD5A1, one SNP showed a P<0.05 by log-rank test for recurrence-free survival and corresponds to a protective allele (rs518673T). Another risk allele for biochemical recurrence (rs166050C) was almost significant (log-rank test=P<0.051) (FIG. 2). The SRD5A1 rs166050 gene polymorphism was associated with an increased recurrence risk of E1R=1.83, 95% CI, 1.04-3.21; P=0.035, while the rs518673 in SRD5A1 was associated with a decreased recurrence risk (HR=0.59, 95% CI, 0.41-0.85; P=0.004). Haplotype analyses further revealed five common SRD5A1 haplotypes (H) with a prevalence of >5% (Table 5). SRD5A1 H2 was significantly associated with the risk of BCR with a HR of 0.64 (0.44-0.94; P=0.023) but did not remained significant in the multivariate model (HR=0.66; CI 95%; 0.46-0.95; p=0.073).

We found 6 htSNPs for the SRD5A2 gene to be associated with recurrence-free survival. The SDR5A2 protective htSNP rs12470143 was significant with a HR of 0.66 (0.46-0.95; P=0.023). A significant association was observed with the non-synonymous SNP V89L (rs523349) with a HR of 2.14 (95% CI, 1.23-3.70; P=0.007) while no association was seen with the other known coding variation A49T (rs9282858) (HR=0.81, 95% CI, 0.36-1.85; P=0.62) (Table 4). The other 4 risk alleles for recurrence were rs2208532, rs2300702, rs4952197, rs676033 with HR of 1.68, 1.88, 1.55 and 1.90, respectively (Table 4). In our Caucasian population, significant linkage disequilibrium was noted between rs676033 and rs523349 (r2=0.90; FIG. 1). Genetic linkage was moderate between rs4952197 and rs676033 (r2=0.69) and between rs49522197 and rs523349 (r2=0.77). The genetic associations between other positive polymorphisms associated with BCR was below 50%. However, genetic linkage between these genetic variations was not ascertained in other populations (such as Asians, African-Americans, Hispanics) and remains to be defined.

For the SRD5A2 gene, we found 4 haplotypes. Of those, H2 and H3 were significantly associated with the risk of BCR but only H3 remained an independent predictor of recurrence in adjusted Cox proportional hazards analysis (adjusted HR=1.63; 1.11-2.39; P=0.013; Table 5).

Protective alleles in both SDR5A genes were significant in the dominant model while risk alleles were significant using the recessive model. Because of the absence of linkage between most htSNPs, the protection conferred by these alleles was not modified by other variants in that population.

TABLE 4
Genotypes frequencies of SNPs in 5α-reductase genes and their association with BCR.

| | | | Genotypes by | | | | | Secondary Model | | | |
|---|---|---|---|---|---|---|---|---|---|---|---|
| | | | categories² | | Genomic Model | | | Model | | | P |
| Genes | SNPs | Genotypes¹ | No BCR | BCR | HR | 95% CI | P value | used | HR | 95% CI | value |
| SRD5A1 | rs518673 | CT | 43/189/159 | 9/50/70 | 0.66 | (0.46-0.94) | 0.023 | Dominant | 0.59 | (0.41-0.85) | 0.004 |
| | | TT | | | 0.52 | (0.26-1.03) | 0.062 | (CT/TT) | | | |
| | rs166050 | TC | 20/152/222 | 14/46/70 | 0.94 | (0.65-1.36) | 0.73 | Recessive | | | |
| | | CC | | | 1.68 | (0.95-2.99) | 0.076 | (CC) | 1.83 | (1.04-3.21) | 0.035 |
| SRD5A2 | rs2208532 | AG | 76/178/140 | 38/55/37 | 1.13 | (0.73-1.74) | 0.60 | Recessive | | | |
| | | GG | | | 1.81 | (1.12-2.91) | 0.016 | (GG) | 1.68 | (1.15-2.48) | 0.008 |
| | rs523349 | GC | 23/139/223 | 15/47/68 | 0.99 | (0.67-1.47) | 0.97 | Recessive | | | |
| | V89L | CC | | | 2.10 | (1.17-3.79) | 0.013 | (CC) | 2.14 | (1.23-3.70) | 0.007 |
| | rs676033 | GA | 31/145/218 | 17/47/66 | 0.99 | (0.66-1.46) | 0.94 | Recessive | | | |
| | | AA | | | 1.82 | (1.04-3.18) | 0.036 | (AA) | 1.88 | (1.12-3.17) | 0.017 |
| | rs12470143 | GA | 90/182/122 | 22/58/50 | 0.62 | (0.41-0.93) | 0.020 | Dominant | 0.66 | (0.46-0.95) | 0.023 |
| | | AA | | | 0.65 | (0.39-1.11) | 0.12 | (GA/AA) | | | |
| | rs2300702 | CG | 71/173/146 | 29/57/43 | 0.84 | (0.55-1.29) | 0.43 | Recessive | | | |
| | | GG | | | 1.40 | (0.85-2.30) | 0.19 | (GG) | 1.55 | (1.01-2.36) | 0.044 |
| | rs4952197 | GA | 20/124/248 | 11/45/74 | 1.09 | (0.74-1.62) | 0.65 | Recessive | | | |
| | | AA | | | 2.11 | (1.10-4.02) | 0.024 | (AA) | 1.90 | (1.01-3.57) | 0.047 |

¹Genotypes shown are for heterozygotes and minor allele homozygotes, as homozygotes for the frequent alleles are the reference (hazard ratio set at 1.00).
²Number of genotypes are as follow: minor allele homozygote/heterozygote/frequent allele homozygote.
HR: hazard ratio;
CI: confidence intervals.
HR: hazard ratio;
95% CI: 95% confidence interval;
BCR: Biochemical recurrence.

TABLE 5
SRD5A haplotypes and their association with risk of BCR.

| | | | Freq | Univariate analysis | | | Multivariate analysis | | |
|---|---|---|---|---|---|---|---|---|---|
| Genes | H | Haplotype sequences | (%) | HR | 95% Cl | P | HR | 95% Cl | P |
| SRD5A1 | 1 | CACATCT | 25.0 | 1.00 | Reference | | 1.00 | Reference | |
| | 2 | TGTGTCT | 21.5 | 0.64 | (0.44-0.94) | 0.023 | 0.70 | (0.48-1.03) | 0.73 |
| | 3 | TACATAC | 20.3 | 0.74 | (0.51-1.07) | 0.111 | 0.81 | (0.55-1.18) | 0.272 |
| | 4 | TGCGTCT | 13.1 | 1.16 | (0.79-1.69) | 0.452 | 1.33 | (0.91-1.96) | 0.144 |
| | 5 | TGTAGAT | 10.4 | 0.77 | (0.49-1.23) | 0.275 | 0.77 | (0.47-1.24) | 0.277 |
| SRD5A2 | 1 | AAAGTACGGGCA | 35.5 | 1.00 | Reference | | 1.00 | Reference | |
| | 2 | GAAGTGGAACCA | 21.0 | 1.42 | (1.02-1.97) | 0.039 | 1.36 | (0.96-1.91) | 0.081 |
| | 3 | GAAGTGCGGGCA | 13.7 | 1.46 | (1.00-2.11) | 0.049 | 1.63 | (1.11-2.39) | 0.013 |
| | 4 | GCGGCAGGGGCG | 12.0 | 1.01 | (0.65-1.56) | 0.977 | 1.00 | (0.64-1.57) | 0.989 |

SRD5A2 SNPs: rs12470143, rs2281546, rs3754838, rs4952222, rs7562326, rs2208532, rs2300702, rs4952197, s676033, rs523349, rs9282858 and rs9332975.
SRD5A1 SNPs: rs166050, rs501999, rs518673, rs3822430, rs500182, rs8192120 and rs4702378.
H: haplotype; Freq: frequencies.

We then investigated the combined effects of protective alleles in both SRD5A genes (Table 6). By combining SRD5A1 (rs518673 T) and SRD5A2 (rs12470143 A), the protective effect was shown to be additive in Kaplan-Meier analysis with the maximum protection conferred by 3 or 4 alleles (FIG. 2C) and remained an independent predictor of recurrence in Cox proportional hazards analysis (HR=0.33, 0.17-0.63; P=0.001 (Table 6). An allele dosage effect was observed with each additional allele diminishing by 26% the risk of recurrence of BCR (P=0.003).

TABLE 6
Combined protective effects of SRD5A1 and SRD5A2 htSNPs.

| Protective | | | | | | | | | |
|---|---|---|---|---|---|---|---|---|---|
| alleles | Cases | Freq | Univariate analysis | | | Multivariate analysis | | | |
| (n) | (n) | (%) | HR | 95% CI | P | HR | 95% CI | P | L-R |
| 0 | 82 | 15.6 | 1.00 | Reference | | 1.00 | Reference | | |
| 1 or 2 | 350 | 66.5 | 0.57 | (0.38-0.86) | 0.008 | 0.46 | (0.30-0.71) | <0.001 | |
| 3 or 4 | 88 | 16.7 | 0.35 | (0.19-0.66) | 0.001 | 0.33 | (0.17-0.63) | 0.001 | 0.005 |
| Continuous¹ | 520 | n/a | 0.75 | (0.63-0.89) | 0.002 | 0.74 | (0.61-0.91) | 0.003 | 0.008 |

¹Number of protective alleles was considered as a continuous variable in univariate and multivariate analysis.
n: number,
L-R: log rank P value.
SRD5A1 protective allele is rs518673T.
SRD5A2 protective allele is rs12470143A.

Using Chi-square likelihood ratio test, no htSNPs were associated with PSA at diagnosis, pathological Gleason score or pathological stage (P>0.05). Only the SRD5A2 rs2208532 was significantly associated with the pathological stage (P=0.048; minor allele homozygote being over-represented in tumors with pT≧T3a). htSNPs associated with BCR were shown to be the third most important predictors of recurrence after the Gleason score and PSA values.

### Discussion

One of the most significant challenges in oncology remains our inability to predict PCa disease recurrence and clinical outcome due to a lack of key prognostic markers. The prediction of recurrence following radical prostatectomy is clearly important for personalized treatments and follow-up strategies due to striking heterogeneity in prostate cancer clinical behaviour. Inherited variations in sex-steroid biosynthesis enzymes are attracting candidates as novel predictive markers since variation in these biotransformation pathways may modify exposure of any residual cancer cells to active androgens produced from adrenal precursors still available after RP in men with PCa.⁴⁷ It is likely that alteration in hormonal exposure of the tumor microenvironment may ultimately also affect the risk of recurrence.

Here, in a cohort of 526 PCa cases, significant associations of BCR with multiple genetic polymorphisms in both SRD5A genes were observed. Of the 19 htSNPs tested, 8 inherited variations (42%) were shown to affect the risk of recurrence after RP either by conferring protection or an elevated risk, independently of known clinical and pathological predictors of prostate cancer recurrence. One of the best characterized SNP in the SRD5A2 gene is the functional V89L (rs523349) polymorphism associated with a decreased in enzymatic activity.^{48, 49} This polymorphism has been associated with an aggressive form of prostate cancer in a recent large case-control study,⁵⁰ with prostate cancer risk in some studies,^{51, 52} and with conflicting results with biochemical recurrence.^{26, 28} The clinical utility of this marker must be further evaluated before any clinical implementation and risk-adapted PCa treatment strategies with these molecular markers. One important finding of our study is a significant association between the V89L (rs523349) polymorphism and the risk of BCR after prostatectomy. A 2.14-fold risk of BCR for homozygotes of the minor C allele was observed. This result is in agreement with Shibata and colleagues²⁸ and with a recent large study that associated this SNP with an aggressive form of PCa.⁵⁰ However, for the SRD5A2 A49T variant (rs9282858), a SNP associated in some studies with the risk of PCa,⁵³ no association was observed with recurrence-free survival after RP indicating that this SNP would have no obvious role in PCa recurrence. This result is consistent with the latest meta-analysis investigating this SNP, showing no association with the risk of PCa.⁵³

Our study is the first to show strong positive associations of multiple independent SRD5A2 genetic variations with BCR. These findings suggest that the 5α-reductase type 2 germline variations play a critical role in prostate cancer recurrence after RP. Most variations represent independent risk alleles for BCR with one variant (rs1247043) associated with significant protection. Our data also argue for a significant role of SRD5A 1 in the risk of recurrence after RP. Variations in this gene have been previously associated with the risk of PCa.^{12, 18} Two SNPs were positively associated with BCR; the rs166050 associated with an increased recurrence risk while the rs518673 conferred protection. By combining the number of protective alleles in both genes, namely SRD5A1 (rs518673T) and SRD5A2 (rs12470143A), this independent protective effect was shown to be additive and maximal in patients carrying 3 to 4 of these alleles.

Strengths of our study includes the large sample size combined with a significant median follow-up time of 7.4 years that provided information on potential confounders. Limitations include the limited number of some clinically relevant events such as metastasis, hormone-resistance, or death related to the localized features of the tumors, that prevented us from looking at the association between molecular signature in SRD5A genes and risk for these events.

Findings are remarkable for the fact that they complement the evidence on somatic and germline genetic changes to predict disease recurrence.^{31, 54-56} Additional investigations are required to characterize the underlying biological mechanisms driving the positive associations of inherited germline variations in the 5α-reductase pathway with BCR. At the time of biochemical relapse and PSA elevation, the disease is particularly androgen-dependent for growth and progression. We can only speculate that these genetic variations influence active androgen formation and exposure of disseminated cancer cells remaining after RP, potentially driving more hormone-dependent cells into cell replication, and subsequently leading to inter-individual differences in recurrence.

In conclusion, our data reveal that multiple genetic markers in SRD5A genes contribute to biochemical recurrence risk after radical prostatectomy. These markers appear independent of current predictors of recurrence such as Gleason score and PSA level and predict risk better than the pathological stage. These findings may ultimately help refine our ability to identify individuals at low or high risk of cancer relapse after RP, beyond known prognostic variables, and for whom a more personalized approach might optimize outcome, especially in the era of 5α-reductase inhibitors therapy.

### REFERENCES

1. Jemal, A. et al. Cancer statistics, 2007. CA Cancer J Clin 57, 43-66 (2007).
2. Gronberg, H. Prostate cancer epidemiology. Lancet 361, 859-64 (2003).
3. Platz, E. A., Rimm, E. B., Willett, W. C., Kantoff, P. W. & Giovannucci, E. Racial variation in prostate cancer incidence and in hormonal system markers among male health professionals. J Natl Cancer Inst 92, 2009-17 (2000).
4. Steinberg, G. D., Carter, B. S., Beaty, T. H., Childs, B. & Walsh, P. C. Family history and the risk of prostate cancer. Prostate 17, 337-47 (1990).
5. Caire, A. A. et al. Delayed prostate-specific antigen recurrence after radical prostatectomy: how to identify and what are their clinical outcomes? Urology 74, 643-7 (2009).
6. D'Amico, A. V. et al. Combination of the preoperative PSA level, biopsy gleason score, percentage of positive biopsies, and MRI T-stage to predict early PSA failure in men with clinically localized prostate cancer. Urology 55, 572-7 (2000).
7. D'Amico, A. V. et al. Utilizing predictions of early prostate-specific antigen failure to optimize patient selection for adjuvant systemic therapy trials. J Clin Oncol 18, 3240-6 (2000).
8. Roberts, S. G., Blute, M. L., Bergstralh, E. J., Slezak, J. M. & Zincke, H. PSA doubling time as a predictor of clinical progression after biochemical failure following radical prostatectomy for prostate cancer. Mayo Clin Proc 76, 576-81 (2001).
9. Belanger, A., Pelletier, G., Labrie, F., Barbier, O. & Chouinard, S. Inactivation of androgens by UDP-glucuronosyltransferase enzymes in humans. Trends Endocrinol Metab. 14, 473-9. (2003).
10. Labrie, F. Adrenal androgens and intracrinology. Semin Reprod Med 22, 299-309 (2004).
11. Labrie, F. Intracrinology. Mol Cell Endocrinol 78, C113-8 (1991).
12. Luu-The, V., Belanger, A. & Labrie, F. Androgen biosynthetic pathways in the human prostate. Best Pract Res Clin Endocrinol Metab 22, 207-21 (2008).
13. Labrie, F. et al. Intracrinology: role of the family of 17 beta-hydroxysteroid dehydrogenases in human physiology and disease. J Mol Endocrinol 25, 1-16 (2000).
14. Vandenput, L. et al. Serum levels of specific glucuronidated androgen metabolites predict BMD and prostate volume in elderly men. J Bone Miner Res 22, 220-7 (2007).
15. Hum, D. W. et al. Characterization of UDP-glucuronosyltransferases active on steroid hormones. J Steroid Biochem Mol Biol 69, 413-23 (1999).
16. Thomas, L. N. et al. Differential alterations in 5alpha-reductase type 1 and type 2 levels during development and progression of prostate cancer. Prostate 63, 231-9 (2005).
17. Elo, J. P. et al. Characterization of 17beta-hydroxysteroid dehydrogenase isoenzyme expression in benign and malignant human prostate. Int J Cancer 66, 37-41 (1996).
18. Montgomery, R. B. et al. Maintenance of intratumoral androgens in metastatic prostate cancer: a mechanism for castration-resistant tumor growth. Cancer Res 68, 4447-54 (2008).
19. Stanbrough, M. et al. Increased expression of genes converting adrenal androgens to testosterone in androgen-independent prostate cancer. Cancer Res. 66, 2815-25. (2006).
20. Titus, M. A. et al. Steroid 5alpha-reductase isozymes I and II in recurrent prostate cancer. Clin Cancer Res 11, 4365-71 (2005).
21. Harris, W. P., Mostaghel, E. A., Nelson, P. S. & Montgomery, B. Androgen deprivation therapy: progress in understanding mechanisms of resistance and optimizing androgen depletion. Nat Clin Pract Urol 6, 76-85 (2009).
22. Horwitz, E. M. Prostate cancer: Optimizing the duration of androgen deprivation therapy. Nat Rev Urol 6, 527-9 (2009).
23. Thompson, I. M. et al. The influence of finasteride on the development of prostate cancer. N Engl J Med 349, 215-24 (2003).
24. Vickers, A. J., Savage, C. J. & Lilja, H. Finasteride to prevent prostate cancer: should all men or only a high-risk subgroup be treated? J Clin Oncol 28, 1112-6.
25. Edwards, S. M. et al. Androgen receptor polymorphisms: association with prostate cancer risk, relapse and overall survival. Int J Cancer 84, 458-65 (1999).
26. Nam, R. K. et al. V89L polymorphism of type-2, 5-alpha reductase enzyme gene predicts prostate cancer presence and progression. Urology 57, 199-204 (2001).
27. Strom, S. S. et al. Androgen receptor polymorphisms and risk of biochemical failure among prostatectomy patients. Prostate 60, 343-51 (2004).
28. Shibata, A. et al. Polymorphisms in the androgen receptor and type II 5 alpha-reductase genes and prostate cancer prognosis. Prostate 52, 269-78 (2002).
29. Celhar, T., Gersak, K., Ovcak, Z., Sedmak, B. & Mlinaric-Rascan, I. The presence of the CYP11A1 (TTTTA)6 allele increases the risk of biochemical relapse in organ confined and low-grade prostate cancer. Cancer Genet Cytogenet 187, 28-33 (2008).
30. Hamada, A. et al. Association of a CYP17 polymorphism with overall survival in Caucasian patients with androgen-independent prostate cancer. Urology 70, 217-20 (2007).
31. Lindstrom, S. et al. Inherited variation in hormone-regulating genes and prostate cancer survival. Clin Cancer Res 13, 5156-61 (2007).
32. Tsuchiya, N. et al. Impact of IGF-I and CYP19 gene polymorphisms on the survival of patients with metastatic prostate cancer. J Clin Oncol 24, 1982-9 (2006).
33. Okugi, H. et al. Association of the polymorphisms of genes involved in androgen metabolism and signaling pathways with familial prostate cancer risk in a Japanese population. Cancer Detect Prey 30, 262-8 (2006).
34. Olsson, M. et al. The UGT2B17 gene deletion is not associated with prostate cancer risk. Prostate 4, 4 (2008).
35. Paris, P. L. et al. Association between a CYP3A4 genetic variant and clinical presentation in African-American prostate cancer patients. Cancer Epidemiol Biomarkers Prey 8, 901-5 (1999).
36. Park, J. et al. Deletion polymorphism of UDP-glucuronosyltransferase 2B17 and risk of prostate cancer in African American and Caucasian men. Cancer Epidemiol Biomarkers Prey. 15, 1473-8. (2006).
37. Park, J. et al. Asp85tyr polymorphism in the udp-glucuronosyltransferase (UGT) 2B15 gene and the risk of prostate cancer. J. Urol. 171, 2484-8. (2004).
38. Park, J. Y. et al. Association between polymorphisms in HSD3B1 and UGT2B17 and prostate cancer risk. Urology. 70, 374-9. (2007).
39. Setlur, S. R. et al. Genetic variation of genes involved in dihydrotestosterone metabolism and the risk of prostate cancer. Cancer Epidemiol Biomarkers Prey 19, 229-39.
40. Cunningham, J. M. et al. Evaluation of genetic variations in the androgen and estrogen metabolic pathways as risk factors for sporadic and familial prostate cancer. Cancer Epidemiol Biomarkers Prey. 16, 969-78. (2007).
41. Cussenot, O. et al. Effect of genetic variability within 8q24 on aggressiveness patterns at diagnosis and familial status of prostate cancer. Clin Cancer Res 14, 5635-9 (2008).
42. Cussenot, O. et al. Combination of polymorphisms from genes related to estrogen metabolism and risk of prostate cancers: the hidden face of estrogens. J Clin Oncol 25, 3596-602 (2007).
43. Mononen, N. & Schleutker, J. Polymorphisms in genes involved in androgen pathways as risk factors for prostate cancer. J Urol 181, 1541-9 (2009).
44. Stephens, M., Smith, N. J. & Donnelly, P. A new statistical method for haplotype reconstruction from population data. Am J Hum Genet. 68, 978-89. (2001).
45. Purcell, S. et al. PLINK: a tool set for whole-genome association and population-based linkage analyses. Am J Hum Genet 81, 559-75 (2007).
46. Barrett, J. C., Fry, B., Maller, J. & Daly, M. J. Haploview: analysis and visualization of LD and haplotype maps. Bioinformatics 21, 263-5 (2005).
47. Labrie, F. et al. Is dehydroepiandrosterone a hormone? J Endocrinol 187, 169-96 (2005).
48. Makridakis, N. et al. A prevalent missense substitution that modulates activity of prostatic steroid 5alpha-reductase. Cancer Res 57, 1020-2 (1997).
49. Makridakis, N. M., di Salle, E. & Reichardt, J. K. Biochemical and pharmacogenetic dissection of human steroid 5 alpha-reductase type II. Pharmacogenetics 10, 407-13 (2000).
50. Cussenot, O. et al. Low-activity V89L variant in SRD5A2 is associated with aggressive prostate cancer risk: an explanation for the adverse effects observed in chemoprevention trials using 5-alpha-reductase inhibitors. Eur Urol 52, 1082-7 (2007).
51. Cicek, M. S. et al. Association of prostate cancer risk and aggressiveness to androgen pathway genes: SRD5A2, CYP17, and the AR. Prostate 59, 69-76 (2004).
52. Salam, M. T., Ursin, G., Skinner, E. C., Dessissa, T. & Reichardt, J. K. Associations between polymorphisms in the steroid 5-alpha reductase type II (SRD5A2) gene and benign prostatic hyperplasia and prostate cancer. Urol Oncol 23, 246-53 (2005).
53. Pearce, C. L. et al. No association between the SRD5A2 gene A49T missense variant and prostate cancer risk: lessons learned. Hum Mol Genet 17, 2456-61 (2008).
54. Sharifi, N., Dahut, W. L. & Figg, W. D. The genetics of castration-resistant prostate cancer: what can the germline tell us? Clin Cancer Res 14, 4691-3 (2008).
55. Ross, R. W. et al. Inherited variation in the androgen pathway is associated with the efficacy of androgen-deprivation therapy in men with prostate cancer. J Clin Oncol 26, 842-7 (2008).
56. Hamada, A. et al. Effect of SLCO1B3 haplotype on testosterone transport and clinical outcome in caucasian patients with androgen-independent prostatic cancer. Clin Cancer Res 14, 3312-8 (2008).

1. An in-vitro method for providing a diagnosis, prognosis or predicting the likelihood of a human subject to develop prostate cancer or a recurrence thereof, said method comprising the steps of:
a) obtaining a nucleic acid from a nucleic acid containing sample from said human subject; and
b) determining the individual's genetic variations (or haplotypes) in SRD5A1 or SRD5A2 gene;
whereby the presence of at least one genetic variation in SRD5A1 or SRD5A2 in said subjects nucleic acid is an indication that said subject has an increased or a decreased likelihood that the prostate cancer will develop or recur.

2. The method of claim 1, wherein step b) comprises the step of:
b′) identifying at least one single nucleotide polymorphism (SNP) in said nucleic acids, said SNP being selected from the group consisting of: rs518673; rs166050; rs12470143; rs2208532; rs2300702; rs4952197 and rs676033 or any of their associated variants as listed in Table 3;
whereby the presence of at least one of said markers in said subjects sample is an indication that said subject has an increased or a decreased likelihood that the prostate cancer will develop or recur.

3. The method of claim 1 or 2, wherein step b) further comprises:
b″) contacting the subjects nucleic acid with a reagent that specifically binds to at least one of said single nucleotide polymorphism (SNP); and
c) detecting the binding of reagent to at least one of said SNP,
whereby the binding of said reagent to at least one SNP is an indication that said subject has an increased or a decreased likelihood that the prostate cancer will develop or recur.

4. The method of claim 3, wherein said SNP is found in reference sequences (rs) selected from the group consisting of: rs166050; rs2208532; rs2300702; rs4952197; and rs676033, or any of their associated SNPs whereby the presence of said SNP is an indication of an increased likelihood that the prostate cancer will develop or recur.

5. The method of claim 4, wherein said SNP is found in reference sequences (rs) selected from the group consisting of: rs2208532 or rs676033, whereby the presence of said SNP is an indication of an increased likelihood that the prostate cancer will develop or recur.

6. The method of claim 5, wherein said SNP is found in rs2208532 or any of its associated SNPs and the presence of said SNP is an indication of an increased likelihood that the prostate cancer will develop or recur.

7. The method of claim 3, wherein said SNP is found in reference sequences (rs) selected from the group consisting of: rs518673 or rs12470143, whereby the presence of said SNP is an indication of a decreased likelihood that the prostate cancer will develop or recur.

8. The method of claim 7, wherein said presence of SNP found in rs518673 or any of its associated SNP is an indication of a decreased likelihood that the prostate cancer will develop or recur.

9. The method of claim 7, wherein the presence of both rs518673T and rs12470143A or any of their associated SNPs is still a further indication of a decreased likelihood that the prostate cancer will develop or recur.

10. The method of claim 1, wherein in step a) said nucleic acid is DNA or RNA.

11. The method of claim 10, wherein said genetic variation of DNA or RNA is detected with the use of a nucleic acid probe.

12. The method of claim 11, wherein said DNA is amplified by PCR prior to incubation with the probe.

13. The method of claim 1, wherein the subjects nucleic acid-containing sample is a tumor or a non-tumor sample.

14. The method of claim 13, wherein the tumor sample originates from a biopsy.

15. The method of claim 13, wherein the subjects non-tumor sample is selected from the group consisting of: tissue or biological fluid.

16. The method of claim 15, wherein the tissue is selected from the group consisting of: lymph node, hair and buccal smear.

17. The method of claim 15, wherein the biological fluid is selected from the group consisting of: sputum, saliva, blood, serum, urine, semen and plasma.

18. A method for adapting a course of treatment of prostate cancer in a human subject after the subject has undergone radical prostatectomy, comprising the steps of:
a) providing a prognosis or predicting the likelihood of a human subject to develop prostate cancer recurrence in accordance with any one of claims 1 to 14; and
b) adapting a course of treatment according to whether said subject has an increased or decreased likelihood that the cancer will recur.

19. The method according to claim 18, wherein, when the likelihood of recurrence is increased, the subject is prescribed 5α-reductase inhibitors therapy.

20. A kit for predicting the likelihood of a human subject to develop prostate cancer recurrence by detecting a SNP in a reference sequence selected from the group consisting of: rs518673; rs166050; rs12470143; rs2208532; rs2300702; rs4952197 and rs676033 or their associated SNPs; said kit comprising reagents for determining the individual's genetic variations (or haplotypes) in SRD5A1 or SRD5A2 gene.

21. The kit of claim 20, comprising PCR primer-probe set, wherein the primer is selected from the group consisting of: SEQ ID Nos. 1 to 38; and the probe is selected from the group consisting of: SEQ ID Nos. 39 to 57.

22. The method of claim 10, wherein said genetic variation of DNA or RNA is detected with the use of a probeless methodology selected from the group consisting of: direct sequencing or pyrosequencing, massively parallel sequencing, high-throughput sequencing high performance liquid chromatography (HPLC) fragment analysis, and capillarity electrophoresis.

23. The method of claim 2, further comprising the step of: identifying the presence of rs523349 (V89L); whereby the presence of rs523349 in said subjects sample is an indication that said subject has an increased likelihood that the prostate cancer will develop or recur.


SEQUENCE LISTING (57 sequences)

SEQ ID NO: 1 - DNA, 30 bp, Artificial Sequence
  Forward Primer
acgttggatgccgtttccctcatgttgttc

SEQ ID NO: 2 - DNA, 30 bp, Artificial Sequence
  Forward Primer
acgttggatgtgtgtattctctggacacgc

SEQ ID NO: 3 - DNA, 30 bp, Artificial Sequence
  Forward Primer
acgttggatgccctccatctttgtagttac

SEQ ID NO: 4 - DNA, 30 bp, Artificial Sequence
  Forward Primer
acgttggatgggtgctatcctagtcccatc

SEQ ID NO: 5 - DNA, 30 bp, Artificial Sequence
  Forward Primer
acgttggatgcccttgatttccaacaccac

SEQ ID NO: 6 - DNA, 30 bp, Artificial Sequence
  Forward Primer
acgttggatgaccaaagtaggttatcgggc

SEQ ID NO: 7 - DNA, 30 bp, Artificial Sequence
  Forward Primer
acgttggatgagccgttacaggtacagaac

SEQ ID NO: 8 - DNA, 30 bp, Artificial Sequence
  Forward Primer
acgttggatgttcttgagctttgttctggg

SEQ ID NO: 9 - DNA, 30 bp, Artificial Sequence
  Forward Primer
acgttggatggggaaaaacgctacctgtgg

SEQ ID NO: 10 - DNA, 30 bp, Artificial Sequence
  Forward Primer
acgttggatgaacagaagacacagcacacc

SEQ ID NO: 11 - DNA, 30 bp, Artificial Sequence
  Forward Primer
acgttggatgtggctactaagaggatagag

SEQ ID NO: 12 - DNA, 30 bp, Artificial Sequence
  Forward Primer
acgttggatggtcacatgagctagttccac

SEQ ID NO: 13 - DNA, 30 bp, Artificial Sequence
  Forward Primer
acgttggatgctctcattaaacctctcccg

SEQ ID NO: 14 - DNA, 30 bp, Artificial Sequence
  Forward Primer
acgttggatgtctacctcagcatatctccc

SEQ ID NO: 15 - DNA, 30 bp, Artificial Sequence
  Forward Primer
acgttggatgaagcacacggagagcctgaa

SEQ ID NO: 16 - DNA, 30 bp, Artificial Sequence
  Forward Primer
acgttggatgtggcacaagcacttccttag

SEQ ID NO: 17 - DNA, 30 bp, Artificial Sequence
  Forward Primer
acgttggatgctgaaaagacagcagaaggg

SEQ ID NO: 18 - DNA, 30 bp, Artificial Sequence
  Forward Primer
acgttggatgaagatatgcccgctgctttg

SEQ ID NO: 19 - DNA, 30 bp, Artificial Sequence
  Forward Primer
acgttggatgacttagctgaggctctcatc

SEQ ID NO: 20 - DNA, 30 bp, Artificial Sequence
  Reverse Primer
acgttggatggagagagaatgagtgctgag

SEQ ID NO: 21 - DNA, 30 bp, Artificial Sequence; identical to SEQ ID NO: 6
  Reverse Primer

SEQ ID NO: 22 - DNA, 30 bp, Artificial Sequence
  Reverse Primer
acgttggatgttgagccctaagagcaacac

SEQ ID NO: 23 - DNA, 30 bp, Artificial Sequence
  Reverse Primer
acgttggatgttccatcaccatgcccattc

SEQ ID NO: 24 - DNA, 30 bp, Artificial Sequence
  Reverse Primer
acgttggatgcattgctgtgaatgcacatc

SEQ ID NO: 25 - DNA, 30 bp, Artificial Sequence; identical to SEQ ID NO: 2
  Reverse Primer

SEQ ID NO: 26 - DNA, 30 bp, Artificial Sequence
  Reverse Primer
acgttggatgtttcttgtttcctaaggtgc

SEQ ID NO: 27 - DNA, 30 bp, Artificial Sequence
  Reverse Primer
acgttggatgacactcttctggaaccactg

SEQ ID NO: 28 - DNA, 30 bp, Artificial Sequence
  Reverse Primer
acgttggatgtgcaggagctgccttccttc

SEQ ID NO: 29 - DNA, 30 bp, Artificial Sequence
  Reverse Primer
acgttggatgtacagtaaaggctcttgccc

SEQ ID NO: 30 - DNA, 30 bp, Artificial Sequence
  Reverse Primer
acgttggatgaagcagcatgtaacctcatc

SEQ ID NO: 31 - DNA, 30 bp, Artificial Sequence
  Reverse Primer
acgttggatgtcttgcctgcaccttttatc

SEQ ID NO: 32 - DNA, 30 bp, Artificial Sequence
  Reverse Primer
acgttggatgtggagaatagattgcggtgg

SEQ ID NO: 33 - DNA, 30 bp, Artificial Sequence
  Reverse Primer
acgttggatggttgcagaaacctcagattg

SEQ ID NO: 34 - DNA, 30 bp, Artificial Sequence
  Reverse Primer
acgttggatgaaggcagctcctgcaggaac

SEQ ID NO: 35 - DNA, 30 bp, Artificial Sequence
  Reverse Primer
acgttggatgcacaaggactgcaattcctg

SEQ ID NO: 36 - DNA, 30 bp, Artificial Sequence
  Reverse Primer
acgttggatgtcctcaggcaaggaaatgtg

SEQ ID NO: 37 - DNA, 30 bp, Artificial Sequence
  Reverse Primer
acgttggatgggccatagtttaccaatccc

SEQ ID NO: 38 - DNA, 30 bp, Artificial Sequence
  Reverse Primer
acgttggatggaggccttgtgtgtttcttc

SEQ ID NO: 39 - DNA, 27 bp, Artificial Sequence
  Probe
ggaaggacgaagcccctaataaaccat

SEQ ID NO: 40 - DNA, 24 bp, Artificial Sequence
  Probe
gaagcaaggaaagctgtgcatcaa

SEQ ID NO: 41 - DNA, 22 bp, Artificial Sequence
  Probe
tgctctcctaagcatatcctac

SEQ ID NO: 42 - DNA, 22 bp, Artificial Sequence
  Probe
ggggcagcttagtaactcatcc

SEQ ID NO: 43 - DNA, 16 bp, Artificial Sequence
  Probe
aatgcacatccagaca

SEQ ID NO: 44 - DNA, 16 bp, Artificial Sequence
  Probe
gctttccttgcttcac

SEQ ID NO: 45 - DNA, 21 bp, Artificial Sequence
  Probe
tctaaggtgcttaatttaccc

SEQ ID NO: 46 - DNA, 25 bp, Artificial Sequence
  Probe
tcaacactgaacaaatcagatcaaa

SEQ ID NO: 47 - DNA, 18 bp, Artificial Sequence
  Probe
cttctgggcctcttctgc

SEQ ID NO: 48 - DNA, 17 bp, Artificial Sequence
  Probe
gcttgacccctcactcc

SEQ ID NO: 49 - DNA, 30 bp, Artificial Sequence
  Probe
ggtctctttgttttacactactcattattt

SEQ ID NO: 50 - DNA, 28 bp, Artificial Sequence
  Probe
cagcaaacgatttccaaggaagtccccc

SEQ ID NO: 51 - DNA, 17 bp, Artificial Sequence
  Probe
ggcggtggagtaaatgt

SEQ ID NO: 52 - DNA, 28 bp, Artificial Sequence
  Probe
tagggtgcttttgttctttttttgtttg

SEQ ID NO: 53 - DNA, 22 bp, Artificial Sequence
  Probe
aaagggaaccaggcggcgcggg

SEQ ID NO: 54 - DNA, 23 bp, Artificial Sequence
  Probe
gggcgtgctgtgctgggcttaga

SEQ ID NO: 55 - DNA, 22 bp, Artificial Sequence
  Probe
gagcaaggaaatgtgaattacc

SEQ ID NO: 56 - DNA, 24 bp, Artificial Sequence
  Probe
ctaaagaaaactatgaatggcgtg

SEQ ID NO: 57 - DNA, 30 bp, Artificial Sequence
  Probe
tagtacttttgttattaacattatatagac